(19) United States
(12) Patent Application Publication
(10) Pub. No.: US 2008/0279771 A1
(43) Pub. Date: Nov. 13, 2008

(54) Novel Imaging Agents for Cancer

(75) Inventors: Grete Mork Kindberg, Oslo (NO); Helge Tolleshaug, Oslo (NO)

Correspondence Address: GE HEALTHCARE, INC., IP DEPARTMENT, 101 CARNEGIE CENTER, PRINCETON, NJ, 08540-6231, US

(73) Assignee: GE HEALTHCARE LIMITED, Buckinghamshire (GB)

(21) Appl. No.: 12/091,804

(22) PCT Filed: Oct. 30, 2006
(86) PCT No.: PCT/GB06/04026

§ 371 Date: Apr. 28, 2008

(30) Foreign Application Priority Data
Oct. 29, 2005 (GB) 0522112.2

(51) Int. Cl. A61K 51/08; C07K 7/06; C07K 7/08; A61K 49/14; C07K 7/50

(52) U.S. Cl. 424/1.69; 530/328; 530/326; 530/317; 530/333; 424/9.1

(57) ABSTRACT

A novel imaging agent is described which comprises a semaphorin moiety and an imaging moiety. The novel imaging agent of the invention may be used in the diagnostic imaging of cancer and in particular, for targeting angiogenesis or metaplasia. Further aspects of the present invention presented herein include a method for the preparation of the imaging agent, a pharmaceutical composition comprising the imaging agent of the invention and a kit for the preparation of said pharmaceutical.

### TECHNICAL FIELD OF THE INVENTION

The present invention relates to in vivo imaging, and more specifically to in vivo imaging of cancer. Novel in vivo imaging agents are provided which target sites of angiogenesis and metaplasia. Also provided are methods for the synthesis of said imaging agents and methods for use of the imaging agents for in vivo imaging.

### DESCRIPTION OF RELATED ART

Semaphorins are a class of biological peptides involved in the development of the nervous and vascular systems. They have been studied largely in the developing nervous system, where they act as repelling cues in axonal guidance. Their expression has also been observed to be altered during angiogenesis and in cancer independently of angiogenesis. Some examples of semaphorins whose expression is of particular interest in the pathophysiology of cancers are semaphorin-3A, semaphorin-3F and semaphorin-4D.

Semaphorin-3A (SEM-3A) binds to the neuropilin-1 receptor (NP-1) resulting in signaling which is essential during the development of the nervous system, but the interaction is not required in vascular system development [Gu et al 2003 Developmental Cell 5: 45-57]. In the vascular system, when the potent blood vessel growth promoter vascular endothelial growth factor (VEGF) binds to the VEGF receptor, NP-1 acts as a co-receptor, enhancing the activity of the VEGF receptor. In prostate tumour cells and breast carcinoma NP-1 is expressed at a rate of about 1-2×10⁵ NP-1 receptors per cell, which is substantially greater than in healthy endothelial cells [Cell 1998 92:735-45]. NP-1 expression also appears to be associated with tumour progression, having been found to correlate positively with disease progression in several human cancers [Adv Exp Med Biol 2002; 515:33-48]. When SEM-3A binds to NP-1, it therefore acts as an inhibitor of VEGF-induced angiogenesis. Binding of SEM-3A to NP-1 on tumour cells has a Kd in the region of 2.8×10⁻¹⁰ M.

In vertebrate embryos, the migration of neural crest cells is guided by signaling induced by binding of semaphorin-3F (SEM-3F) to the neuropilin-2 receptor (NP-2). As with SEM-3A/NP-1 signaling, SEM-3F/NP-2 signaling is not required in vascular system development but SEM-3F expression, like SEM-3A expression, is known to be altered in certain pathophysiological processes. Many tumour cells express SEM-3F, but its down-regulation is associated with highly metastatic tumours [Bielenberg et al 2004 J. Clin. Invest. 114: 1260-71]. When SEM-3F is over-expressed in metastatic melanoma cells, it completely inhibits spontaneous metastasis to lymph nodes and lungs. The resulting tumours are highly encapsulated and poorly vascularized suggesting that SEM-3F may be an endothelial cell chemorepulsant that inhibits tumour angiogenesis [Klagsbrun & Eichmann, Cytokine & Growth Factor Reviews 2005; 16:535-48]. Due to the resultant paucity of blood vessels, SEM-3F may also block metastasis.

Semaphorin-4D has been observed to be angiogenic both in vivo and in vitro, and the effect is mediated by its high-affinity receptor, plexin-B1 (PX-B1) [Conrotto et al 2005 Blood 105(11): 4321-9]. Binding of SEM-4D to PX-B1 stimulates the tyrosine kinase activity of the receptor for hepatocyte growth factor, Met, resulting in tyrosine phosphorylation of both receptors and invasive growth of epithelial cells [Giordano et al 2002 Nat. Cell Biol. 4 720-724].

Semaphorins are also known from the patent literature. WO 03/102584 discloses semaphorin-like polypeptides having anti-angiogenic properties. The proteins are used to inhibit angiogenesis, cell migration and actin filament formation. In vitro diagnostic applications are discussed in relation to use of antibodies against the polypeptides of the invention for the detection of said polypeptides, e.g. by ELISA. It is suggested that detection may be facilitated by coupling the antibody to a detectable substance. There is no discussion that the semaphorin-like polypeptides themselves might be detectably labeled for any purpose.

U.S. Pat. No. 6,800,273 discloses peptide compounds linked to an imaging moiety having affinity for receptors which are upregulated in angiogenesis. A semaphorin receptor, NP-1, is disclosed as one of the receptors of interest. However, there is no mention of any other semaphorin receptors, or of using a semaphorin compound as the targeting moiety. WO 01/91805 and Perret et a also disclose peptide compounds that target NP-1 linked to an imaging moiety, but again neither of these documents mentions using a semaphorin compound as the targeting moiety.

There therefore remains a need for improved imaging agents for imaging angiogenesis that also target other pathophysiological aspects of cancer.

### SUMMARY OF THE INVENTION

Novel imaging agents are described which comprise a semaphorin moiety and an imaging moiety. The novel imaging agent of the invention may be used in the diagnostic imaging of cancer and in particular, for targeting angiogenesis or metaplasia. Further aspects of the present invention presented herein include a method for the preparation of the imaging agent, a pharmaceutical composition comprising the imaging agent of the invention and a kit for the preparation of said pharmaceutical composition.

### DETAILED DESCRIPTION OF THE INVENTION

In a first aspect, the present invention comprises an imaging agent comprising;
(i) a semaphorin moiety; and
(ii) an imaging moiety
wherein said imaging moiety is either an integral part of the semaphorin moiety or is conjugated to the semaphorin moiety via a suitable chemical group.

By the term “imaging agent” is meant a compound designed to target a particular physiology or pathophysiology in a mammal, and which can be detected following its administration to the mammalian body in vivo.

A “semaphorin moiety” of the present invention is a synthetic peptide or small molecule compound which shares structural similarity with a semaphorin and has affinity for a semaphorin receptor. The term “affinity” in the context of the present invention is defined as the ability to inhibit SEM-3A-induced growth cone collapse of dorsal root ganglion neurons in vitro [Luo et a/1995 Neuron 14 1131-40] at IC₅₀ values of between 10 μM and 100 μM, preferably between 1 μM and 10 μM, most preferably between 100 nM and 1 μM, especially preferably between 100 nM and 10 nM and most especially preferably between 10 nM and 0.01 nM. Preferred semaphorin receptors of the invention are NP-1, NP-2 and Px-B1.

Semaphorin moieties of the present invention which are peptides can range in size from 5-mer peptides to 800-mer peptides (i.e. peptides comprising 5 to 800 amino acids). Preferably, the peptides of the present invention are 5- to 100-mer peptides, most preferably 5- to 50-mer peptides and most especially preferably 5- to 20-mer peptides. The peptides may be cyclic or linear or combinations thereof. The peptides may be of synthetic or natural origin, but are preferably synthetic.

By the term “cyclic peptide” is meant a sequence of 5 to 15 amino acids in which the two terminal amino acids are bonded together by a covalent bond which may be a peptide or disulphide bond or a synthetic non-peptide bond such as a thioether, phosphodiester, disiloxane or urethane bond.

By the term “amino acid” is meant an L- or D-amino acid, amino acid analogue or amino acid mimetic which may be naturally occurring or of purely synthetic origin, and may be optically pure, i.e. a single enantiomer and hence chiral, or a mixture of enantiomers. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

Preferred peptides of the invention are SEM-3A, SEM-3F and SEM-4D, as well as analogues and peptide fragments thereof. The sequence of human SEM-3A (Swiss-Prot Q14563-http://www.expasy.org/uniprot/Q14563) in its unprocessed precursor form consists of 771 amino acids. The sequence of human SEM-3F (Swiss-Prot Q13275-http://www.expasy.org/uniprot/Q13275) in its unprocessed precursor form consists of 785 amino acids. The sequence of human SEM-4D (Swiss-Prot Q92854-http://www.expasy.org/uniprot/Q92854) in its unprocessed precursor form consists of 862 amino acids.

Particularly preferred peptide fragments of SEM-3A and SEM-3F that show inhibition of semaphorin-induced growth cone collapse are reported by Williams et al [J. Neurochem. 2005 92 1180-90]:

Peptide sequence

| | |
|---|---|
| 1 | N-Ac-HAVEHGFMQTLLKVTLE-NH₂ |
| 2 | N-Ac-QTLLKVTLE-NH₂ |
| 3 | N-Ac-CAVEHGFMC-NH₂ |
| 4 | N-Ac-CEHGFMQC-NH₂ |
| 5 | N-Ac-CFQRRNEC-NH₂ |

Sequences 3-5 represent disulphide constrained cyclic peptides, i.e. having Cys-Cys bonds.

The ligand-binding face of SEM-4D has been characterised by Love et al [Nat. Sruct. Biol. 10(10) 843-8]. Small peptide sequences derivable from this sequence are also suitable as semaphorin moieties of the present invention.

Synthetic peptides of the invention may be obtained by conventional solid phase synthesis, as described by Merrifield employing an automated peptide synthesizer (J. Am. Chem. Soc., 85: 2149 (1964)).

The semaphorin moiety can also be a “small molecule compound”, which is a non-peptide compound having binding characteristics similar to the parent peptide. For example, Kikuchi et of disclosed the isolation and characterization of a SEM-3A binding inhibitor, xanthofulvin:

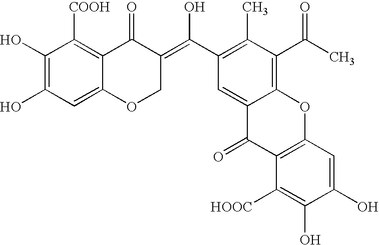

In a method for the synthesis of the imaging agent of the invention, a precursor compound is reacted with a suitable source of the imaging moiety. The precursor compound is a derivative of one of the above semaphorin moieties having a chemical group capable of reacting with the suitable source of the imaging moiety. This is discussed in further detail below in relation to the second aspect of the invention.

The “imaging moiety” may be detected either external to the human body or via use of detectors designed for use in vivo, such as intravascular radiation or optical detectors such as endoscopes, or radiation detectors designed for intra-operative use.

The “imaging moiety” is preferably chosen from:
(i) a radioactive metal ion;
(ii) a paramagnetic metal ion;
(iii) a gamma-emitting radioactive halogen;
(iv) a positron-emitting radioactive non-metal;
(v) a hyperpolarized NMR-active nucleus;
(vi) a reporter suitable for in vivo optical imaging;
(vii) a β-emitter suitable for intravascular detection.

Most preferred imaging moieties are those which can be detected by either nuclear imaging or optical imaging. Especially preferred imaging moieties are radioactive, especially radioactive metal ions, gamma-emitting radioactive halogens and positron-emitting radioactive non-metals, particularly those suitable for imaging using SPECT or PET.

When the imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸ Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ²⁴¹I. Preferred positron-emitting radioactive non-metals are ¹¹C, ¹³N, ¹⁸F and ¹²⁴I, especially ¹¹C and ¹⁸F, most especially ¹⁸F.

When the imaging moiety is a hyperpolarized NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarized” is meant enhancement of the degree of polarisation of the NMR-active nucleus over its' equilibrium polarisation. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarized. At least one carbon atom of the compounds which accumulates at sites of active thrombosis in viva is suitably enriched with ¹³C, which is subsequently hyperpolarized.

When the imaging moiety is a reporter suitable for in vivo optical imaging, the reporter is any moiety capable of detection either directly or indirectly in an optical imaging procedure. The reporter might be a light scatterer (e.g. a coloured or uncolored particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, indoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared (NIR) region, between 400 nm and 3 μm, particularly between 600 and 1300 nm. Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

When the imaging moiety is a β-emitter suitable for intravascular detection, suitable such β-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³Br.

Preferred imaging agents of the invention do not undergo facile metabolism in vivo, and hence most preferably exhibit a half-life in vivo of 60 to 240 minutes in humans. The imaging agent is preferably excreted via the kidney (i.e. exhibits urinary excretion). The imaging agent preferably exhibits a signal-to-background ratio at diseased foci of at least 1.5, most preferably at least 5, with at least 10 being especially preferred. Where the imaging agent comprises a radioisotope, clearance of one half of the peak level of imaging agent which is either non-specifically bound or free in vivo, preferably occurs over a time period less than or equal to the radioactive decay half-life of the radioisotope of the imaging moiety.

The molecular weight of the imaging agent is preferably up to 5000 Daltons. Most preferably, the molecular weight is in the range 150 to 3000 Daltons, most especially preferably 200 to 1500 Daltons, with 300 to 800 Daltons being ideal.

In a second aspect, the present invention provides a method for the preparation of the imaging agent of the invention comprising reaction of:
(i) a precursor; and,
(ii) a suitable source of an imaging moiety,
   wherein said precursor is a derivative of the semaphorin moiety of the invention.

The “precursor” must be designed so that chemical reaction with a convenient chemical form of the imaging moiety occurs site-specifically; can be conducted in the minimum number of steps (ideally a single step); and without the need for significant purification (ideally no further purification), to give the desired imaging agent. Such precursors are synthetic and can conveniently be obtained in good chemical purity. The “precursor” may optionally comprise a protecting group for certain functional groups of the semaphorin moiety.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Preferably, the precursor of the invention is a semaphorin moiety derivatised with a chemical group which:
(i) is capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution;
(iv) comprises a derivative containing an aromatic ring activated towards nucleophilic or electrophilic substitution;
(v) comprises a derivative containing a functional group which undergoes facile alkylation; or
(vi) comprises a derivative which alkylates thiol-containing compounds to give a thioether-containing product.

When the imaging moiety comprises a metal ion, the precursor comprises a “ligand”, which is a chemical group capable of complexing the metal ion to form a metal complex. By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include the semaphorin moiety itself plus other excipients in the preparation in vitro (e.g. radioprotectants or antimicrobial preservatives used in the preparation), or endogenous compounds in vivo (e.g. glutathione, transferrin or plasma proteins).

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:

(i) diaminedioximes of Formula (X):

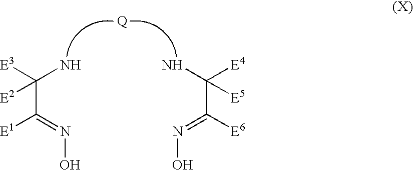

where E¹-E⁶ are each independently an R* group;
each R* is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more R* groups together with the atoms to which they are attached form a carbocyclic, heterocyclic, saturated or unsaturated ring, and wherein one or more of the R* groups is conjugated to the semaphorin moiety;
and Q is a bridging group of formula -(J)_{f}-;
where f is 3, 4 or 5 and each J is independently —O—, —NR*- or —C(R*)₂— provided that -(J)_{f}-contains a maximum of one J group which is —O— or —NR*-.

Preferred Q groups are as follows:

Q=-(CH₂)(CHR*)(CH₂)— i.e. propyleneimine oxime or PnAO derivatives;
Q=-(CH₂)₂(CHR*)(CH₂)₂— i.e. pentyleneamine oxime or PentAO derivatives;

### Q=-(CH₂)₂NR*(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

The semaphorin moiety is preferably conjugated at either the E¹ or E⁶ R* group, or an R* group of the Q moiety. Most preferably, it is conjugated to an R* group of the Q moiety. When it is conjugated to an R* group of the Q moiety, the R* group is preferably at the bridgehead position. In that case, Q is preferably —(CH₂)(CHR*)(CH₂)—, —(CH₂)₂(CHR*)(CH₂)₂— or —(CH₂)₂NR*(CH₂)₂—, most preferably —(CH₂)₂(CHR*)(CH₂)₂—. An especially preferred bifunctional diaminedioxime chelator has the Formula (Z):

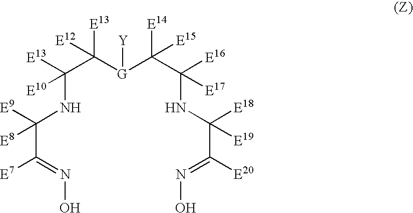

where:
E⁷-E²⁰ are each independently an R group;

### G is N or CR;

Y is -(A)ₙ- where:
-(A)ₙ- is a linker group where each A is independently —CO—, —CR₂—, —CR═CR—, —C≡C—, —CR₂CO₂—, —CO₂CR₂—, —NR—, —NRCO—, —CONR, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group or a polyalkyleneglycol, polylactic acid or polyglycolic acid moiety;
n is an integer of value 0 to 10;
each R group is independently H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, or 2 or more R groups, together with the atoms to which they are attached form a carbocyclic, heterocyclic, saturated or unsaturated ring.

A preferred chelator of Formula (Z) is of Formula (Za):

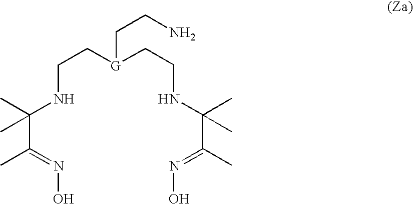

where G is as defined above and is preferably CH (chelate Z);
such that the semaphorin moiety is conjugated via the bridgehead —CH₂CH₂NH₂ group.

Further suitable chelators of the invention include:

(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam or dioxocyclam.
(v) N₂O₂ ligands having a diaminediphenol donor set.

The above-described ligands are particularly suitable for complexing technetium e.g. ^{94m}Tc or ^{99m}Tc, and are described more fully by Jurisson et al [Chem. Rev., 99, 2205-2218 (1999)]. The ligands are also useful for other metals, such as copper (⁶⁴Cu or ⁶⁷Cu), vanadium (e.g. ⁴⁸V), iron (e.g. ⁵²Fe), or cobalt (e.g. ⁵⁵Co). Other suitable ligands are described in Sandoz WO 91/01144, which includes ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. When the radiometal ion is technetium, the ligand is preferably a chelating agent which is tetradentate. Preferred chelating agents for technetium are the diaminedioximes, or those having an N₂S₂ or N₃S donor set as described above.

It is envisaged that the role of the linker group -(A)ₙ- is to distance the relatively bulky technetium complex, which results upon metal coordination, from the active site of the semaphorin moiety so that e.g. receptor binding is not impaired. This can be achieved by a combination of flexibility (e.g. simple alkyl chains), so that the bulky group has the freedom to position itself away from the active site and/or rigidity such as a cycloalkyl or aryl spacer which orientates the metal complex away from the active site. The nature of the linker group can also be used to modify the biodistribution of the resulting technetium complex of the conjugate. Thus, e.g. the introduction of ether groups in the linker will help to minimise plasma protein binding, or the use of polymeric linker groups such as polyalkyleneglycol, especially PEG (polyethyleneglycol) can help to prolong the lifetime of the agent in the blood in vivo.

Preferred linker groups -(A)ₙ- have a backbone chain (i.e. the linked atoms which make up the -(A)ₙ- moiety) which contains 2 to 10 atoms, most preferably 2 to 5 atoms, with 2 or 3 atoms being especially preferred. A minimum linker group backbone chain of 2 atoms confers the advantage that the aza-diaminedioxime chelator is well-separated from the biological targeting moiety so that any interaction is minimised. Furthermore, the semaphorin moiety groups are unlikely to compete effectively with the coordination of the chelator to the metal ion. In this way, both the biological targeting characteristics of the semaphorin moiety, and the metal complexing capability of the chelator are maintained. It is strongly preferred that the semaphorin moiety is bound to the chelator in such a way that the linkage does not undergo facile metabolism in blood. That is because such metabolism would result in the imaging metal complex being cleaved off before the labelled semaphorin moiety reaches the desired in vivo target site. The semaphorin moiety is therefore preferably covalently bound to the metal complexes of the present invention via -(A)ₙ- linker groups which are not readily metabolised. Suitable such linkages are carbon-carbon bonds, amide bonds, urea or thiourea linkages, or ether bonds.

Non-peptide linker groups such as alkylene groups or arylene groups have the advantage that there are no significant hydrogen bonding interactions with the conjugated semaphorin moiety so that the linker does not wrap round onto the semaphorin moiety. Preferred alkylene spacer groups are —(CH₂)_{q}— where q is an integer of value 2 to 5. Preferably q is 2 or 3. Preferred arylene spacers are of formula:

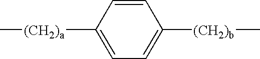

where: a and b are each independently 0, 1 or 2.

A preferred Y group is thus —CH₂CH₂-(A)ₚ-,—where p is an integer of value 0 to 3.

When the semaphorin moiety is a peptide, Y is preferably —CH₂CH₂-(A)ₚ- where -(A)ₚ- is —CO— or —NR—. When -(A)ₚ- is —NH—, this grouping has the additional advantage that it stems from the symmetrical intermediate N(CH₂CH₂NH₂)₃, which is commercially available.

When the imaging metal is technetium, the usual technetium starting material is pertechnetate, i.e. TcO₄— which is technetium in the Tc(VII) oxidation state. Pertechnetate itself does not readily form complexes, hence the preparation of technetium complexes usually requires the addition of a suitable reducing agent such as stannous ion to facilitate complexation by reducing the oxidation state of the technetium to the lower oxidation states, usually Tc(I) to Tc(V). The solvent may be organic or aqueous, or mixtures thereof. When the solvent comprises an organic solvent, the organic solvent is preferably a biocompatible solvent, such as ethanol or DMSO. Preferably the solvent is aqueous, and is most preferably isotonic saline.

When the imaging moiety comprises a radioactive halogen, such as iodine, the precursor suitably comprises the following reactive groups: a non-radioactive precursor halogen atom such as an aryl iodide or bromide (to permit radioiodine exchange); an activated aryl ring (e.g. phenol or aniline groups); an imidazole ring; an indole ring; an organometallic compound (e.g. trialkyltin or trialkylsilyl); or an organic compound such as triazene or a good leaving group for nucleophilic substitution such as an iodonium salt. Methods of introducing radioactive halogens (including ¹²³I and ¹⁸F) are described by Bolton [J. Lab. Comp. Radiopharm., 45, 485-528 (2002)]. Examples of suitable aryl groups to which radioactive halogens, especially iodine can be attached are given below:

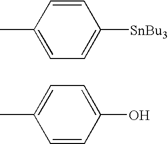

Both contain substituents which permit facile radioiodine substitution onto the aromatic ring. Alternative substituents containing radioactive iodine can be synthesised by direct iodination via radiohalogen exchange, e.g.:

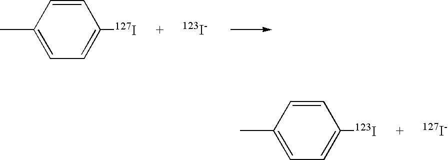

When the imaging moiety comprises a radioactive isotope of iodine the radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine. An iodine atom bound to an activated aryl ring like phenol has also, under certain circumstances, been observed to have limited in vivo stability.

When the imaging moiety comprises a radioactive halogen, such as ¹²³I and ¹⁸F, the precursor preferably comprises a functional group that will react selectively with a radiolabelled synthon and thus upon conjugation gives the imaging agent of the invention. By the term “radiolabelled synthon” is meant a small, synthetic organic molecule which is:
(i) already radiolabelled such that the radiolabel is bound to the synthon in a stable manner;
(ii) comprises a functional group designed to react selectively and specifically with a corresponding functional group which is part of the desired compound to be radiolabelled.

This approach gives better opportunities to generate imaging agents with improved in vivo stability of the radiolabel relative to direct radiolabelling approaches.

A synthon approach also allows greater flexibility in the conditions used for the introduction of the imaging moiety.

Examples of precursors suitable for the generation of imaging agents of the present invention are those which comprise an aminoxy group, a thiol group, an amine group, a maleimide group or an N-haloacetyl group. A preferred method for selective labelling is to employ aminoxy derivatives as precursors, as taught by Poethko et al [J. Nuc. Med., 45, 892-902 (2004)]. Such precursors are then condensed with a radiohalogenated-benzaldehyde synthon under acidic conditions (e.g. pH 2 to 4), to give the desired radiohalogenated imaging agent via a stable oxime ether linkage. The precursor therefore preferably comprises an aminoxy group of formula —NH(C═O)CH₂—O—NH₂. Another preferred method of labelling is when the precursor comprises a thiol group which is alkylated with radiohalogenated maleimide-containing synthon under neutral conditions (pH 6.5-7.5) e.g. as taught by Toyokuni et al [Bioconj. Chem. 14, 1253-1259 (2003)] to label thiols.

An additional preferred method of labelling is when the precursor comprises an amine group which is condensed with the synthon N-succinimidyl 4-[¹²³I]iodobenzoate at pH 7.5-8.5 to give amide bond linked products. The use of N-hydroxysuccinimide ester to label peptides is taught by Vaidyanathan et al [Nucl. Med. Biol., 19(3), 275-281 (1992)] and Johnstrom et al [Clin. Sci., 103 (Suppl. 48), 45-85 (2002)].

When the imaging moiety comprises a radioactive isotope of fluorine the radiofluorine atom may form part of a fluoroalkyl or fluoroalkoxy group, since alkyl fluorides are resistant to in vivo metabolism. Radiofluorination may be carried out via direct labelling using the reaction of ¹⁸F-fluoride with a suitable precursor having a good leaving group, such as an alkyl bromide, alkyl mesylate or alkyl tosylate. Alternatively, the radiofluorine atom may be attached via a direct covalent bond to an aromatic ring such as a benzene ring. For such aryl systems, the precursor suitably comprises an activated nitroaryl ring, an aryl diazonium salt, or an aryl trialkylammonium salt. The direct radiofluorination of biomolecules is, however, often detrimental to sensitive functional groups since these nucleophilic reactions are carried out with anhydrous [¹⁸F]fluoride ion in polar aprotic solvents under strong basic conditions. Some precursors may exhibit instability under basic conditions. Therefore direct radiofluorination of precursors of the imaging agent of the present invention is not a preferred labelling method. Examples of preferred methods for radiofluorination involve the use of radiolabelled synthons that are conjugated selectively to precursors of the invention, as discussed above for the labelling of radiohalogens in general.

¹⁸F can also be introduced by N-alkylation of amine precursors with alkylating agents such as ¹⁸F(CH₂)₃OMs (where Ms is mesylate) to give N—(CH₂)₃¹⁸F, O-alkylation of hydroxyl groups with ¹⁸F(CH₂)₃OMs, ¹⁸F(CH₂)₃OTs or ¹⁸F(CH₂)₃Br or S-alkylation of thiol groups with ¹⁸F(CH₂)₃OMs or ¹⁸F(CH₂)₃Br. ¹⁸F can also be introduced by alkylation of N-haloacetyl groups with a ¹⁸F(CH₂)₃OH reactant, to give —NH(CO)CH₂O(CH₂)₃¹⁸F derivatives or with a ¹⁸F(CH₂)₃SH reactant, to give —NH(CO)CH₂S(CH₂)₃¹⁸F derivatives. 18F can also be introduced by reaction of maleimide-containing precursors with ¹⁸F(CH₂)₃SH. For aryl systems, ¹⁸F-fluoride nucleophilic displacement from an aryl diazonium salt, an aryl nitro compound or an aryl quaternary ammonium salt are suitable routes to aryl-¹⁸F labelled synthons useful for conjugation to precursors of the imaging agent.

Precursors that comprises a primary amine group can also be labelled with ¹⁸F by reductive amination using ¹⁸F—C₆H₄—CHO as taught by Kahn et al [J. Lab. Comp. Radiopharm. 45, 1045-1053 (2002)] and Borch et al [J. Am. Chem. Soc. 93, 2897 (1971)]. This approach can also usefully be applied to aryl primary amines, such as compounds comprising phenyl-NH₂ or phenyl-CH₂NH₂ groups.

An especially preferred method for ¹⁸F-labelling is when the precursor comprises an aminoxy group of formula —NH(C═O)CH₂—O—NH₂ which is condensed with ¹⁸F—C₆H₄—CHO under acidic conditions (e.g. pH 2 to 4). This method is particularly useful for precursors which are base-sensitive.

Further details of synthetic routes to ¹⁸F-labelled derivatives are described by Bolton, J. Lab. Comp. Radiopharm., 45, 485-528 (2002).

The precursor may optionally be supplied covalently attached to a solid support matrix. In that way, the desired imaging agent product forms in solution, whereas starting materials and impurities remain bound to the solid phase. Precursors for solid phase electrophilic fluorination with ¹⁸F-fluoride are described in WO 03/002489. Precursors for solid phase nucleophilic fluorination with ¹⁸F-fluoride are described in WO 03/002157. The solid support-bound precursor may therefore be provided as a kit cartridge which can be plugged into a suitably adapted automated synthesizer. The cartridge may contain, apart from the solid support-bound precursor, a column to remove unwanted fluoride ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customer requirements for radioactive concentration, volumes, time of delivery etc. Conveniently, all components of the kit are disposable to minimise the possibility of contamination between runs and will be sterile and quality assured.

In a third aspect, the present invention provides a pharmaceutical composition which comprises the imaging agent as described above, together with a biocompatible carrier, in a form suitable for mammalian administration. In a preferred embodiment, the pharmaceutical composition is a radiopharmaceutical composition.

The “biocompatible carrier” is a fluid, especially a liquid, in which the imaging agent is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilize more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. In the case of radiopharmaceutical compositions, the pre-filled syringe may optionally be provided with a syringe shield to protect the operator from radioactive dose. Suitable such radiopharmaceutical syringe shields are known in the art and preferably comprise either lead or tungsten.

The pharmaceutical compositions of the present invention may be prepared from kits, as is described in the fourth embodiment below. Alternatively, the pharmaceutical compositions may be prepared under aseptic manufacture conditions to give the desired sterile product. They may also be prepared under non-sterile conditions, followed by terminal sterilisation using e.g. gamma-irradiation, autoclaving, dry heat or chemical treatment (e.g. with ethylene oxide). Preferably, the pharmaceutical compositions of the present invention are prepared from kits.

As described above in relation to the first embodiment, for radiopharmaceutical compositions the most preferred radioactive imaging moieties of the invention are ^{99m}Tc, ¹²³I and ¹⁸F.

In a fourth aspect, the present invention provides kits for the preparation of the pharmaceutical compositions of the third embodiment using the method of the second embodiment. Such kits therefore comprise a suitable precursor as described above in relation to the second aspect of the invention, preferably in sterile non-pyrogenic form, so that reaction with a sterile source of an imaging moiety gives the desired pharmaceutical with the minimum number of manipulations. Such considerations are particularly important for radiopharmaceuticals, in particular where the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

Suitable kit containers comprise a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

Preferred aspects of the precursor when employed in the kit are as described for the second embodiment above. The precursors for use in the kit may be employed under aseptic manufacture conditions to give the desired sterile, non-pyrogenic material. The precursors may also be employed under non-sterile conditions, followed by terminal sterilisation using e.g. gamma-irradiation, autoclaving, dry heat or chemical treatment (e.g. with ethylene oxide). Preferably, the precursors are employed in sterile, non-pyrogenic form. Most preferably the sterile, non-pyrogenic precursors are employed in the sealed container as described above.

The precursor of the kit is preferably supplied covalently attached to a solid support matrix as described above in relation to the second embodiment.

For ^{99m}Tc, the kit is preferably lyophilised and is designed to be reconstituted with sterile ^{99m}Tc-pertechnetate (TcO₄—) from a ^{99m}Tc radioisotope generator to give a solution suitable for human administration without further manipulation. Suitable kits comprise a container (e.g. a septum-sealed vial) containing the uncomplexed chelating agent, together with a pharmaceutically acceptable reducing agent such as sodium dithionite, sodium bisulphite, ascorbic acid, formamidine sulphinic acid, stannous ion, Fe(II) or Cu(I); together with at least one salt of a weak organic acid with a biocompatible cation. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The kits for preparation of ^{99m}Tc imaging agents may optionally further comprise a second, weak organic acid or salt thereof with a biocompatible cation, which functions as a transchelator. The transchelator is a compound which reacts rapidly to form a weak complex with technetium, then is displaced by the chelator of the kit. This minimises the risk of formation of reduced hydrolysed technetium (RHT) due to rapid reduction of pertechnetate competing with technetium complexation. Suitable such transchelators are the weak organic acids and salts thereof described above, preferably tartrates, gluconates, glucoheptonates, benzoates, or phosphonates, preferably phosphonates, most especially diphosphonates. A preferred such transchelator is MDP, i.e. methylenediphosphonic acid, or a salt thereof with a biocompatible cation.

As an alternative to use of a chelator in free form, the kit for preparation of ^{99m}Tc imaging agents may optionally contain a non-radioactive metal complex of the chelator which, upon addition of the technetium, undergoes transmetallation (i.e. ligand exchange) giving the desired product. Suitable such complexes for transmetallation are copper or zinc complexes.

The pharmaceutically acceptable reducing agent used in the kit is preferably a stannous salt such as stannous chloride, stannous fluoride or stannous tartrate, and may be in either anhydrous or hydrated form. The stannous salt is preferably stannous chloride or stannous fluoride.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. The “biocompatible cation” and preferred embodiments thereof are as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the in vivo imaging agent product itself. The antimicrobial preservative may, however, also optionally be used to inhibit the growth of potentially harmful micro-organisms in one or more components of the kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the precursor is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The imaging agent of the invention is useful for in vivo imaging. Accordingly, in a fifth aspect, the present invention provides an imaging agent of the invention for use in an in vivo diagnostic or imaging method, e.g. SPECT, PET or optical imaging. Preferably said method relates to the in vivo imaging of cancer and therefore has utility in the diagnosis of cancer.

This aspect of the invention also provides a method for the in vivo diagnosis or imaging of cancer in a subject, comprising administration of a pharmaceutical composition of the third aspect of the invention. Said subject is preferably a mammal and most preferably a human. In an alternative embodiment, this aspect of the invention furthermore provides for the use of the imaging agent of the invention for imaging cancer in vivo in a subject wherein said subject is previously administered with the pharmaceutical composition of the third aspect of the invention.

By “previously administered” is meant that the step involving the clinician, wherein the pharmaceutical is given to the patient e.g., intravenous injection, has already been carried out. This aspect of the invention also encompasses use of the imaging agent of the first embodiment for the manufacture of diagnostic agent for the diagnostic imaging in vivo of cancer.

Furthermore, this aspect of the invention provides for use of the imaging agent of the invention in the manufacture of a pharmaceutical for the in vivo diagnosis or imaging of cancer.

In a preferred embodiment of this aspect of the invention, the diagnostic imaging of cancer comprises imaging either of the processes of angiogenesis or metaplasia.

In a sixth aspect the invention provides a method of monitoring the effect of treatment of a human or animal body with a drug to combat cancer, said method comprising administering to said body a compound of the invention and detecting the uptake of said compound, said administration and detection optionally but preferably being effected repeatedly, e.g. before, during and after treatment with said drug.

The evaluation of therapeutic intervention in cancer patients with measurable disease has several applications:
the evaluation of the anti-neoplastic activity of new anti-cancer drugs;
to determine efficacious therapeutic regimens;
the identification of the optimal dose and dosing schedules for new anticancer drugs;
the identification of optimal dose and dosing schedules for existing anticancer drugs and drug combinations;
the more efficient stratification of cancer patients in clinical trials into responders and non-responders of therapeutic regimens;
the efficient and timely evaluation of response of individual patients to established therapeutic anti-cancer regimens.

The invention is illustrated by the non-limiting Examples detailed below.

### BRIEF DESCRIPTION OF THE EXAMPLES

Example 1 describes the synthesis of the chelate of Formula Za (where G=CH, i.e. chelate Z), as mentioned in the description.

Example 2 and 3 describe the synthesis of precursor compounds of the invention.

Example 4 describes ^{99m}Tc labeling of the precursor compounds of Examples 2 and 3.

The following abbreviations are used:

DIEA=N-Ethyl-N-(1-methylethyl)-2-Propanamine (Hunig's base)
DMF=N,N′-dimethylformamide
HATU=O-(7-Azabenzotriazole-1-yl)-N,N,N′N′-tetramethyluronium hexafluorophosphate
HPLC=high-performance liquid chromatography
ITLC=instant thin-layer chromatography
LC-MS=liquid chromatography-mass spectrometry
MS=mass spectrometry
NMR=nuclear magnetic resonance
RF=retention factor
RP-HPLC=reverse-phase high-performance liquid chromatography
THF=tetrahydrofuran
TLC=thin-layer chromatography

### EXAMPLES

### Example 1

### Synthesis of Chelate Z

### Step a

### Preparation of tris(methyloxycarbonylmethyl)methane

3-(methoxycarbonylmethylene)glutaric acid dimethylester (89 g, 267 mmol) in methanol (200 ml) was shaken with (10% palladium on charcoal: 50% water) (9 g) under an atmosphere of hydrogen gas (3.5 bar) for (30 h). The solution was filtered through kieselguhr and concentrated in vacuo to give 3-(methoxycarbonylmethyl)glutaric acid dimethylester as an oil, yield (84.9 g, 94%). NMR ¹H(CDCl₃), δ 2.48 (6H, d, J=8 Hz, 3×CH₂), 2.78 (1H, hextet, J=8 Hz CH,) 3.7 (9H, s, 3×CH₃).

NMR ¹³C(CDCl₃), δ 28.6, CH; 37.50, 3×CH₃; 51.6, 3×CH₂; 172.28, 3×COO.

### Step b

### Amidation of trimethylester with p-methoxy-benzylamine

Tris(methyloxycarbonylmethyl)methane [2 g, 8.4 mmol] was dissolved in p-methoxy-benzylamine (25 g, 178.6 mmol). The apparatus was set up for distillation and heated to 120° C. for 24 hrs under nitrogen flow. The progress of the reaction was monitored by the amount of methanol collected. The reaction mixture was cooled to ambient temperature and 30 ml of ethyl acetate was added, then the precipitated triamide product stirred for 30 min. The triamide was isolated by filtration and the filter cake washed several times with sufficient amounts of ethyl acetate to remove excess p-methoxy-benzylamine. After drying 4.6 g, 100%, of a white powder was obtained. The highly insoluble product was used directly in the next step without further purification or characterisation.

### Step c

### Preparation of 1,1,1-tris[2-(p-methoxybenzylamino)ethyl]methane

To a 1000 ml 3-necked round bottomed flask cooled in a ice-water bath the triamide from step 2(a) (10 g, 17.89 mmol) is carefully added to 250 ml of 1M borane solution (3.5 g, 244.3 mmol) borane. After complete addition the ice-water bath is removed and the reaction mixture slowly heated to 60° C. The reaction mixture is stirred at 60° C. for 20 hrs. A sample of the reaction mixture (1 ml) was withdrawn, and mixed with 0.5 ml 5N HCl and left standing for 30 min. To the sample 0.5 ml of 50 NaOH was added, followed by 2 ml of water and the solution was stirred until all of the white precipitate dissolved. The solution was extracted with ether (5 ml) and evaporated. The residue was dissolved in acetonitrile at a concentration of 1 mg/ml and analysed by MS. If mono- and diamide (M+H/z=520 and 534) are seen in the MS spectrum, the reaction is not complete. To complete the reaction, a further 100 ml of 1M borane THF solution is added and the reaction mixture stirred for 6 more hrs at 60° C. and a new sample withdrawn following the previous sampling procedure. Further addition of the 1M borane in THF solution is continued as necessary until there is complete conversion to the triamine.

The reaction mixture is cooled to ambient temperature and 5N HCl is slowly added, [CARE: vigorous foam formation occurs!]. HCl was added until no more gas evolution is observed. The mixture was stirred for 30 min and then evaporated. The cake was suspended in aqueous NaOH solution (20-40%; 1:2 w/v) and stirred for 30 minutes. The mixture was then diluted with water (3 volumes). The mixture was then extracted with diethylether (2×150 ml) [CARE: do not use halogenated solvents]. The combined organic phases were then washed with water (1×200 ml), brine (150 ml) and dried over magnesium sulphate. Yield after evaporation: 7.6 g, 84% as oil.

NMR ¹H(CDCl₃), δ: 1.45, (6H, m, 3×CH₂; 1.54, (1H, septet, CH); 2.60 (6H, t, 3×CH₂N); 3.68 (6H, s, ArCH₂); 3.78 (9H, s, 3×CH₃O); 6.94 (6H, d, 6×Ar). 7.20 (6H, d, 6×Ar).

NMR ¹³C(CDCl₃), δ: 32.17, CH; 34.44, CH₂; 47.00, CH₂; 53.56, ArCH₂; 55.25, CH₃O; 113.78, Ar; 129.29, Ar; 132.61; Ar; 158.60, Ar.

### Step d

### Preparation of 1,1,1-tris(2-aminoethyl)methane

1,1,1-tris[2-(p-methoxybenzylamino)ethyl]methane (20.0 gram, 0.036 mol) was dissolved in methanol (100 ml) and Pd(OH)₂ (5.0 gram) was added. The mixture was hydrogenated (3 bar, 100° C., in an autoclave) and stirred for 5 hours. Pd(OH)₂ was added in two more portions (2×5 gram) after 10 and 15 hours respectively. The reaction mixture was filtered and the filtrate was washed with methanol. The combined organic phase was evaporated and the residue was distilled under vacuum

(1×10⁻², 110° C.) to give 2.60 gram (50%) of 1,1,1-tris(2-aminoethyl)methane.

NMR ¹H(CDCl₃), δ 2.72 (6H, t, 3×CH₂N), 1.41 (H, septet, CH), 1.39 (6H, q, 3×CH₂).

NMR ¹³C(CDCl₃), δ 39.8 (CH₂NH₂), 38.2 (CH₂.), 31.0 (CH).

### Step e

### Preparation of Formula Za (where G=C)

To a solution of tris(2-aminoethyl)methane (4.047 g, 27.9 mmol) in dry ethanol (30 ml) was added potassium carbonate anhydrous (7.7 g, 55.8 mmol, 2 eq) at room temperature with vigorous stirring under a nitrogen atmosphere. A solution of 3-chloro-3-methyl-2-nitrosobutane (7.56 g, 55.8 mol, 2 eq) was dissolved in dry ethanol (100 ml) and 75 ml of this solution was dripped slowly into the reaction mixture. The reaction was followed by TLC on silica [plates run in dichloromethane, methanol, concentrated (0.88 sg) ammonia; 100/30/5 and the TLC plate developed by spraying with ninhydrin and heating]. The mono-, di- and tri-alkylated products were seen with RF's increasing in that order. Analytical HPLC was run using RPR reverse phase column in a gradient of 7.5-75% acetonitrile in 3% aqueous ammonia. The reaction was concentrated in vacuo to remove the ethanol and resuspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate 12 eq, 4.3 g, 55.8 mmol) to ensure good chromatography. The aqueous solution was stored at 4° C. overnight before purifying by automated preparative HPLC.

Yield (2.2 g, 6.4 mmol, 23%).

Mass spec; Positive ion 10 V cone voltage. Found: 344; calculated M+H=344.

NMR ¹H(CDCl₃), δ 1.24 (6H, s, 2×CH₃), 1.3 (6H, s, 2×CH₃), 1.25-1.75 (7H, m, 3×CH₂, CH), (3H, s, 2×CH₂), 2.58 (4H, m, CH₂N), 2.88 (2H, t CH₂N₂), 5.0 (6H, s, NH₂, 2×NH, 2×OH).

NMR ¹H((CD₃)₂SO) δ 1.14×CH; 1.29, 3×CH₂; 2.1 (4H, t, 2×CH₂);

NMR ¹³C((CD₃)₂SO), δ 9.0 (4×CH₃), 25.8 (2×CH₃), 31.0 2×CH₂, 34.6 CH₂, 56.8 2×CH₂N; 160.3; C═N.

HPLC conditions: flow rate 8 ml/min using a 25 mm PRP column

A=3% ammonia solution (sp.gr=0.88)/water; B=Acetonitrile

| Time | % B |
|---|---|
| 0 | 7.5 |
| 15 | 75.0 |
| 20 | 75.0 |
| 22 | 7.5 |
| 30 | 7.5 |

Load 3 ml of aqueous solution per run, and collect in a time window of 12.5-13.5 min.

### Example 2

### Synthesis of Peptide Precursors for ^{99m}Tc-Labelling

### Prophetic Example

Synthesis of the peptides 1-5 mentioned in the description will be carried out by the methods described by Williams et al [J. Neurochem. 2005 92 1180-90].

These peptides will be conjugated at either their amino or unprotected carboxy terminus to the chelator of Formula Za via the bridgehead —CH₂CH₂NH₂ group in order to form a precursor compound.

Briefly, the protected peptides will be coupled with the chelator of Formula Za in solution using Benzotriazole-1-yl-oxytris-pyrrolidino-phosphonium hexafluorophosphate and 1-hydroxybenzotriazole as the coupling agents. Precursor compounds will be obtained by deprotection in reagent K (reagent K is 82.5% TFA, 5% phenol, 5% processed water, 5% thioanisole, 2.5% ethanedithiol). Where required, purification will be by RP-HPLC using TFA followed by a second purification and salt exchange with acetic acid, lyophilisation, filtration with a 0.22μ filter and a final lyophilisation to give purified precursor compounds.

### Example 3

### Synthesis of Xanthofulvin Precursor for ^{99m}Tc-Labelling

### Prophetic Example

Xanthofulvin will be obtained by fermentation and purification, as described by Kikuchi et alp [J. Biol. Chem. 2003 278(44) 42985-91].

Xanthofulvin will be conjugated at either of its carboxyl groups to the chelator of Formula Za via the bridgehead —CH₂CH₂NH₂ group in order to form a precursor compound.

To a solution of xanthofulvin (9 μmol) in DMF will be added the chelator of Formula Za (9 μmol), HATU (Applied Biosystems, 9 μmol) and DIEA (Fluka, 18 μmol). After 20 min the reaction time the mixture was concentrated and the residue will be purified by preparative HPLC (column Phenomenex Luna C18(2) 5 μm 21.2×250 mm, solvents: A=water/0.1% TFA and B=acetonitrile/0.1% TFA; gradient 10-80% B over 60 min; flow 10.0 ml/min, UV detection at 214 nm), giving 4.2 mg (43%) of lyophilised product. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 ml/min, UV detection at 214 nm, ESI-MS; t_{R}=4.17 min, m/z 1082.5 (MH⁺)) and NM R spectroscopy will confirm the structure.

### Example 4

### ^{99m}Tc-Labelling of the Precursor Compounds of Examples 2 and 3

### Prophetic Example

For ^{99m}Tc labelling, 50 μg of a precursor compound will be added to a nitrogen filled vial and dissolved in 50 μL water, 150 μL of sodium gluconate solution (25 mg in 6 mL H₂O), 100 μL ammonium acetate (pH 4.0, 50 mM), 1 mL TcO₄ soln (500 MBq) and 50 μL SnCl₂ soln (20 mg in 100 mL H₂0). The mixture will be analysed by ITLC and HPLC.

What is claimed is:

1.-30. (canceled)

31. An imaging agent comprising;
(i) a semaphorin moiety which is a 5 to 100-mer peptide fragment derived from a semaphorin; and
(ii) an imaging moiety,
wherein said imaging moiety is either an integral part of the semaphorin moiety or is conjugated to the semaphorin moiety via a suitable chemical group.

32. The imaging agent of claim 31 wherein said peptide fragment is derived from SEM-3A, SEM-3F or SEM-4D.

33. The imaging agent of claim 32 wherein said peptide fragment is selected from:
(i) N—Ac—HAVEHGFMQTLLKVTLE-NH₂;
(ii) N—Ac-QTLLKVTLE-NH₂;
(iii) N—Ac—CAVEHGFMC-NH₂;
(iv) N—Ac—CEHGFMQC-NH₂;
(v) N—Ac—CFQRRNEC-NH₂,
wherein sequences (iii) to (v) are cyclic peptides.

34. The imaging agent of claim 31 wherein said imaging moiety is selected from:
(i) a radioactive metal ion;
(ii) a gamma-emitting radioactive halogen;
(iii) a positron-emitting radioactive non-metal.

35. The imaging agent of claim 34 wherein said imaging moiety is a radioactive metal ion selected from ⁶⁴Cu, ⁴⁸V, ⁵² Fe, ⁵⁵Co, ^{94m}Tc, ⁶⁸Ga, ^{99m}Tc, ¹¹¹In, ^{113m}In and ⁶⁷Ga.

36. The imaging agent of claim 35 wherein said radioactive metal ion is ^{99m}Tc.

37. The imaging agent of claim 34 wherein said imaging moiety is a gamma-emitting radioactive halogen selected from ¹²³I, ¹³¹I and ⁷⁷Br.

38. The imaging agent of claim 37 wherein said gamma-emitting radioactive halogen is ¹²³I.

39. The imaging agent of claim 34 wherein said imaging agent is a positron-emitting radioactive non-metal selected from ¹¹C, ¹³N, ¹⁵O, ¹⁷F, ¹⁸F, ⁷⁵Br, ⁷⁶Br and ¹²⁴I.

40. The imaging agent of claim 39 wherein said positron-emitting radioactive non-metal is ¹⁸F.

41. A method for the preparation of an imaging agent comprising;
(i) a semaphorin moiety which is a 5 to 100-mer peptide fragment derived from a semaphorin; and
(ii) an imaging moiety,
wherein said imaging moiety is either an integral part of the semaphorin moiety or is conjugated to the semaphorin moiety via a suitable chemical group further comprising reaction of:
(a) a precursor; and,
(b) a suitable source of said imaging moiety,
wherein said precursor is a derivative of said semaphorin moiety.

42. The method according to claim 41 wherein said precursor comprises a chemical group which:
(i) is a chelator capable of complexing a metallic imaging moiety;
(ii) comprises an organometallic derivative such as a trialkylstannane or a trialkylsilane;
(iii) comprises a derivative containing an alkyl halide, alkyl tosylate or alkyl mesylate for nucleophilic substitution;
(iv) comprises a derivative containing an aromatic ring activated towards nucleophilic or electrophilic substitution;
(v) comprises a derivative containing a functional group which undergoes facile alkylation; or
(vi) comprises a derivative which alkylates thiol-containing compounds to give a thioether-containing product.

43. A pharmaceutical composition comprising the imaging agent of claim 31, together with a biocompatible carrier in a form suitable for mammalian administration.

44. A kit for the preparation of the pharmaceutical composition of claim 43 via the method for the preparation of an imaging agent comprising;
(i) a semaphorin moiety which is a 5 to 100-mer peptide fragment derived from a semaphorin; and
(ii) an imaging moiety,
wherein said imaging moiety is either an integral part of the semaphorin moiety or is conjugated to the semaphorin moiety via a suitable chemical group further comprising reaction of:
(c) a precursor; and,
(d) a suitable source of said imaging moiety,
wherein said precursor is a derivative of said semaphorin moiety.

45. The imaging agent of claim 31 for use in an in vivo diagnostic or imaging method.

46. The imaging agent of claim 45 wherein said in vivo diagnostic or imaging method relates to the in vivo imaging of cancer.

47. A method for the in vivo diagnosis or imaging of cancer in a subject, comprising administration of the pharmaceutical composition of claim 43.

48. A method of monitoring the effect of treatment of a human or animal body with a drug to combat cancer, said method comprising administering to said body the imaging agent of claim 31 and detecting the uptake of said compound, said administration and detection optionally but preferably being effected repeatedly, e.g. before, during and after treatment with said drug.
